(19) United States
(12) Patent Application Publication
(10) Pub. No.: US 2023/0415128 A1
(43) Pub. Date: Dec. 28, 2023

(54) AIR-CLEANING DEVICE AND AIR-CLEANING METHOD

(71) Applicant: PURESPACE INC., Busan (KR)

(72) Inventors: Jeong Se YUN, Busan (KR); Jae Sung LEE, Busan (KR); Hae Young JEONG, Busan (KR); Sun Young LEE, Seoul (KR)

(73) Assignee: PURESPACE INC., Busan (KR)

(21) Appl. No.: 18/036,649

(22) PCT Filed: Nov. 12, 2020
(86) PCT No.: PCT/KR2020/015851

§ 371 Date: May 12, 2023

(51) Int. Cl. B01J 23/34; A61L 9/015; B01J 35/02; B01J 37/03; B01J 37/10; B01J 37/02

(52) U.S. Cl. CPC B01J 23/34; A61L 9/015; B01J 35/026; B01J 35/023; B01J 37/033; B01J 37/10; B01J 37/0215

(57) ABSTRACT

Provided are an air-cleaning device and method for reducing harmful gas including ethylene and harmful microorganisms, and an air-cleaning system including the air-cleaning device.

### TECHNICAL FIELD

The present disclosure relates to an air-cleaning device and method for reducing harmful gas including ethylene and harmful microorganisms, and an air-cleaning system including the air-cleaning device.

### BACKGROUND ART

It is important to remove organic substances from the air in aspects of maintenance of the freshness of fruits and vegetables, as well as health and accident prevention through a decrease in harmful substances. This is because, after plant growth and harvest, harmful gases including ethylene, which are closely involved in the growth of crops, and harmful microorganisms such as airborne microbe, fungi, and bacteria, spread through the air.

In order to reduce or remove harmful gases including ethylene and harmful microorganisms, various attempts have been made using filters, adsorption, and chemical reactions. Among them, chemical reactions that can function with high efficiency for a long time have been usually utilized. Such chemical reactions are performed based on such a mechanism that oxygen-based radicals and ozone with high oxidizing power are used to remove organic substances and the remaining ozone is removed, and then, clean air is discharged into the atmosphere. However, even with the mechanism, the concentration of ozone could not be reduced for a long time and efficiently so as not to harm crops and workers.

Therefore, there is still a need for an air-cleaning device, an air-cleaning method, and an air-cleaning system including the air-cleaning device, which effectively reduce or remove harmful gases including ethylene, harmful microorganisms, and ozone.

### DISCLOSURE OF INVENTION

### Technical Problem

One aspect is to provide a novel air-cleaning device.

Another aspect is to provide an air-cleaning method that not only reduces or removes harmful gases including ethylene and harmful microorganisms, but also decomposes ozone.

Another aspect is to provide an air-cleaning system including the air-cleaning device.

### Solution to Problem

According to one aspect, an air-cleaning device for reducing harmful gases including ethylene and harmful microorganisms, the air-cleaning device including:

an air inlet for intaking air from the outside;

an ozone generating unit in which at least one ozone generator of a corona discharge ozone generator or a cold plasma ozone generator is located;

an ozone decomposition unit for removing ozone generated in the ozone generating unit, in which a support and an ozone decomposition catalyst structure including a nano manganese oxide are located; and

an air outlet for outflowing the internal air to the outside, wherein

the nano manganese oxide may be located on at least a portion of the inside and the surface of the support,

the nano manganese oxide may include at least one of α-MnO₂, β-MnO₂, γ-MnO₂, δ-MnO₂, or amorphous MnO₂, and

the nano manganese oxide may have a shape selected from a nanorod shape, a nanofiber shape, a nano sea-urchin shape, a nanoflower shape, or a nanosheet shape.

The manganese oxide includes at least one of α-MnO₂ or β-MnO₂, and

α-MnO₂ or β-MnO₂ has a nanorod shape, a nanofiber shape, or a nano sea-urchin shape, and an aspect ratio of 1:5 to 1:1000.

The nano manganese oxide may be β-MnO₂, and

the δ-MnO₂ may have a nanoflower shape or a nanosheet shape, and a thickness thereof may be from 5 nm to 400 nm.

In an embodiment, the nano manganese oxide may be a crystalline MnO₂ nanoparticle or amorphous MnO₂ nanoparticle, and

the nanoparticle may have a diameter of 1 nm to 500 nm.

The nano manganese oxide may further include nano manganese oxide doped with a

transition metal in an amount of 0.01 wt % to 50 wt % based on the total weight of the nano manganese oxide.

The nano manganese oxide may further include at least one selected from metal oxide, silicon oxide, carbon nanotubes, activated carbon, graphene, or graphene oxide.

The support may be a ceramic material, a metal material, or a combination of these, in the form of a monolith or a foam.

The ozone decomposition catalyst structure may be a binder-free structure.

The air-cleaning device enables the inflow and outflow of air in one direction.

At least one fan may be provided in at least one of the air inlet and the air outlet.

The harmful gas may include organic-inorganic harmful gas including ethylene, ammonia, acetaldehyde, or a combination thereof.

The harmful microorganisms may include fungi, E. coli, Pseudomonas aeruginosa, Staphylococcus, or a combination of these.

According to another aspect, provided is an air-cleaning method of reducing harmful gases including ethylene and harmful microorganisms, the air-cleaning method including:

a first step of reducing harmful gas including ethylene and harmful microorganisms in air by using at least one ozone generator of a corona discharge ozone generator or a cold plasma ozone generator; and

a second step of decomposing ozone generated in the first step, by using an ozone decomposition catalyst structure including a support and a nano manganese oxide located in at least a portion of the inside and surface of the support, wherein

the nano manganese oxide may include at least one of α-MnO₂, β-MnO₂, γ-MnO₂, δ-MnO₂, or amorphous MnO₂, and

the nano manganese oxide may have a shape selected from a nanorod shape, a nanofiber shape, a nano sea-urchin shape, a nanoflower shape, or a nanosheet shape.

According to another aspect,

provided is an air-cleaning system including the air-cleaning device.

### Advantageous Effects of Invention

The air-cleaning device, the air-cleaning method, and the air-cleaning system, according to the present disclosure, are effectively reduce or remove harmful gas including ethylene, harmful microorganism, and ozone, without exchanging filters. Furthermore, the air-cleaning device, the air-cleaning method, and the air-cleaning system, according to the present disclosure, may maintain the freshness of fruits and vegetables in a closed space such as a reservoir.

### BRIEF DESCRIPTION OF DRAWINGS

FIG. 1 shows a schematic diagram showing an ozone decomposition catalyst structure according to an embodiment.

FIG. 2 shows a schematic diagram of an air-cleaning device according to an embodiment.

FIG. 3 shows a schematic diagram of an air-cleaning device according to an embodiment.

FIG. 4 shows X-ray diffraction (XRD) experiment results of an α-MnO₂ nanorod catalyst of an ozone decomposition catalyst structure prepared according to Preparation Example 1.

FIGS. 5 to 9 show scanning electron microscope (SEM) images of ozone decomposition catalysts of the ozone decomposition catalyst structures manufactured according to Preparation Example 1, Preparation Example 2, Preparation Example 3, Preparation Example 4, and Preparation Example 5.

FIG. 10 shows an evaluation result of ethylene gas reduction performance of air-cleaning devices manufactured according to Example 1, Comparative Example 1, and Comparative Example 2.

FIG. 11 shows an evaluation result of ozone decomposition performance of the air-cleaning devices manufactured according to Example 1 and Comparative Example 2.

### MODE FOR THE INVENTION

Hereinafter, with reference to the attached drawings, an air-cleaning device and an air-cleaning method for reducing harmful gas including ethylene and harmful microorganisms, and an air-cleaning system including the air-cleaning device, according to an example embodiments will be described in detail. The following embodiments are provided as an example, and do not limit the present disclosure, and the present disclosure is defined only by the claims to be described later. In addition, in this specification and the drawings, elements having substantially the same functions are denoted by the same reference numeral, and redundant explanations thereof will be omitted.

Unless defined otherwise, all technical and scientific terms used herein have the same meaning as commonly understood by one of ordinary skill in the art to which the present disclosure belongs. In case of conflicts in the meanings, the definition in the present specification, including definitions, will be preferred.

Although methods and materials similar or equivalent to those described herein may be used in the practice or testing of the present invention, suitable methods and materials are described herein. Singular expressions include plural expressions unless the context clearly indicates otherwise.

In the present specification, terms such as “include” or “have” are intended to indicate the existence of features, numbers, processes, operations, components, parts, components, materials, or combinations thereof described in the specification. It is to be understood that the possibility of the presence or addition of one or more other features, numbers, processes, operations, components, parts, components, materials, or combinations thereof is not preliminarily excluded.

The term “combinations thereof” used herein refers to a mixture, alloy or combination with one or more of the described components.

The term “and/or” used herein refers to any combination or all combinations of at least one constituent element of a list. The term “or” used herein refers to “and/or”. The terms “at least one type”, “one or more types”, or “one or more” before components may supplement the list of all constituent elements, and may not supplement individual constituent elements of the recited list.

The term “aspect ratio” used herein refers to “a ratio of a shorter length (or width) to a longer length” or “a ratio of diameter to length” according to the shape, unless defined otherwise.

The term “diameter” or “average diameter (D50)” refers to a diameter or average diameter (D50) based on the assumption that the shape is a sphere or a shape close to the sphere, unless specified otherwise. However, when the shape is not a sphere or a shape close to the sphere, the length (width) of the shorter axis is defined as a diameter.

In the drawings, the thickness is enlarged or reduced in order to clearly express various layers and regions. Like reference numerals denote like elements throughout the specification. Throughout the specification, when a component such as a layer, film, region, plate, or the like is described to be “on” or “above” another component, this includes not only the case in which one component is directly on another component, but also the case in which an intervening layer is placed between the components. Throughout the specification, terms such as first and second may be used to describe various elements, but the elements should not be limited by the terms. The terms are used only for the purpose of distinguishing one component from another.

An air-cleaning device for reducing harmful gases including ethylene and harmful microorganisms according to an embodiment includes: an air inlet for intaking air from the outside; an ozone generating unit in which at least one ozone generator of a corona discharge ozone generator or a cold plasma ozone generator is located; an ozone decomposition unit for removing ozone generated in the ozone generating unit in which a support and an ozone decomposition catalyst structure including an nano manganese oxide are located; and an air outlet for outflowing the internal air to the outside, wherein the nano manganese oxide may be located on at least a portion of the inside and the surface of the support, the nano manganese oxide may include at least one of α-MnO₂, β-MnO₂, γ-MnO₂, δ-MnO₂, or amorphous MnO₂, and the nano manganese oxide may have a shape selected from a nanorod shape, a nanofiber shape, a nano sea-urchin shape, or a nanoflower shape.

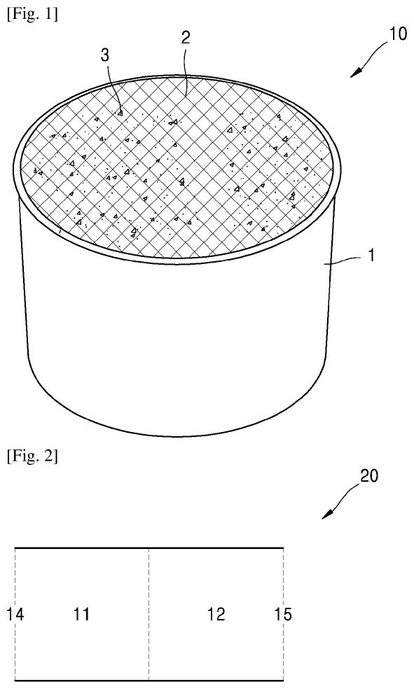

FIG. 1 shows a schematic diagram showing an ozone decomposition catalyst structure 10 according to an embodiment.

Referring to FIG. 1, the ozone decomposition catalyst structure 10 according to an embodiment includes a support 1, and a nano manganese oxide 3 located in an inside 2 or/and on the surface of the support 1.

The ozone decomposition catalyst structure 10 according to the present embodiment may include the nano manganese oxide 3 as a catalyst. From among transition metal oxides, the nano manganese oxide 3 actively generates reactive oxygen species required to oxidize harmful gases including ethylene by ozone at a low temperature of 100° C. or less. In an embodiment, reactive oxygen species are produced by decomposition of ozone. Compared to other transition metal oxides, the nano manganese oxide 3 has oxygen vacancies enough to generate reactive oxygen species required for decomposition of ozone. Therefore, the nano manganese oxide 3 has higher ozone decomposition activity compared to other transition metal oxides.

In an embodiment, the nano manganese oxide 3 may include at least one of α-MnO₂ or β-MnO₂, α-MnO₂ or β-MnO₂ may have a nanorod shape, nanofiber or a nano sea-urchin shape, and an aspect ratio of 1:5 to 1:1000. In an embodiment, the aspect ratio of a nanorod shape, a nanofiber shape, and a nano sea-urchin shape may be from 1:5 to 1:100, from 1:5 to 1:90, from 1:5 to 1:80, from 1:5 to 1:70, from 1:5 to 1:60, from 1:5 to 1:50, from 1:5 to 1:40, or from 1:5 to 1:30.

In an embodiment, the nano manganese oxide 3 may be α-MnO₂, and the α-MnO₂ may have a nanorod shape or a nano sea-urchin shape. Since α-MnO₂ has more oxygen vacancies to generate reactive oxygen species required for the ozone decomposition, α-MnO₂ has excellent ozone decomposition catalytic activity compared to manganese oxide having other crystal structures.

For example, the manufacturing method of α-MnO₂ is as follows. At room temperature, a manganese chloride (MnCl₂·4H₂O) aqueous solution, a manganese acetate (Mn(CH₃COO)₂·4H₂O) aqueous solution, or a manganese sulfate (MnSO₄·5H₂O) aqueous solution, which are used as a starting material, is reacted with a predetermined equivalent amount of KMnO₄ to precipitate MnO₂, which is then reacted in a hydrothermal reactor at a temperature of 180° C. for 12 hours. In addition, in order to increase the yield or/and purity of the α-MnO₂ catalyst, the manufacturing method may be performed several times.

When the aspect ratio of the nanorod shape, the nanofiber shape, or the nano sea-urchin shape is within the range, the specific surface area is large and thus, excellent catalytic activity may be obtained, and the coating may be performed in a binder-free form.

In an embodiment, the nano manganese oxide 3 may be β-MnO₂, the β-MnO₂ may have the nanoflower shape or the nanosheet form, and may have the thickness of 5 nm to 100 nm. In an embodiment, the thickness of the nano manganese oxide 3 may be from 5 nm to 90 nm, from 5 nm to 80 nm, from 5 nm to 70 nm, from 5 nm to 60 nm, or from 5 nm to 50 nm. Only within these ranges, the nano manganese oxide 3 has a large specific surface area with respect to the weight thereof, and thus, excellent catalytic activity may be obtained.

In an embodiment, the nano manganese oxide 3 may be a crystalline MnO₂ or amorphous MnO₂ nanoparticle, and the nanoparticle may have a diameter of 1 nm to 500 nm. The diameter range of the nanoparticles refers to the diameter range of the short-axis length (or width). In an embodiment, the diameter of the nanoparticles may be from 1 nm to 450 nm, from 1 nm to 400 nm, from 1 nm to 350 nm, from 1 nm to 300 nm, from 1 nm to 250 nm, or from 1 nm to 200 nm. The nano manganese oxide may be easily coated using a coating solution containing the nano manganese oxide 3 having these diameter ranges, and, after the coating, the nano manganese oxide 3 is not separated from the support 1 and catalytic activity may be maintained high.

The nano manganese oxide 3 may further include nano manganese oxide doped with a transition metal in an amount of 0.01 wt % to 50 wt % based on the total weight of the nano manganese oxide. In an embodiment, the nano manganese oxide 3 may further include nano manganese oxide doped with 0.01 wt % to 45 wt % of transition metal, 0.01 wt % to 40 wt % of transition metal, 0.01 wt % to 35 wt % of transition metal, or 0.01 wt % to 30 wt % of transition metal, based on the total weight of the nano manganese oxide. Examples of the doped transition metal include copper, cerium, iron, cobalt, and nickel. However, the transition metal is not limited thereto, and any transition metal that is available in the related art may be used for the doping. The nano manganese oxide 3 doped with such a transition metal may effectively maintain the catalytic activity of ozone decomposition for a long time even under a high humidity environment.

The nano manganese oxide 3 may further include at least one selected from metal oxide, silicon oxide, carbon nanotubes, activated carbon, graphene, or graphene oxide. Examples of the metal oxide include copper oxide, cerium oxide, cobalt oxide, nickel oxide, or aluminum oxide. However, the metal oxide is not limited thereto, and any metal oxide that is available in the related art may be used. Examples of the carbon nanotubes may include single-walled carbon nanotubes (SWCNT), multi-walled carbon nanotubes (MWCNT), or a combination of these. The nano manganese oxide 3 further including at least one selected from metal oxide, silicon oxide, carbon nanotubes, activated carbon, graphene, or graphene oxide has high affinity for harmful gases including ethylene, and thus can effectively adsorb the harmful gases. Therefore, the nano manganese oxide may have a higher catalytic activity of ozone decomposition.

The support 1 may be a ceramic material, a metal material, or a combination of these, in the form of a monolith or a foam. Examples of the metal material include stainless steel or aluminum. However, the metal oxide is not limited thereto, and any metal material that is available in the related art may be used.

In an embodiment, the support 1 may be a porous support. In an embodiment, the support 1 may be a porous inorganic-material support. In an embodiment, the support 1 may be a monolith.

In an embodiment, the porous inorganic-material support may include a porous ceramic material containing 50% or more of MgO, SiO₂, and Al₂O₃. The porous ceramic material may have a ceramic honeycomb structure. The porous ceramic material may have, per inch, about 100 to about 500, for example, about 200 to about 500, for example, about 300 to about 400 square cells. In the porous ceramic material, air or the like may be introduced through the square cells.

The porous ceramic material may increase a catalytic activity due to a high strength and a large specific surface area. In addition, the porous ceramic material has good ventilation and thus, may reduce pressure loss, and maintains the shape thereof even by external environments such as strong acids, high temperatures, and strong winds.

The porous ceramic material may have a cross section having various shapes such as a circular shape, an oval shape, a rectangular shape, or a square shape. The porous ceramic material may have the structure of a cylinder, a rectangular parallelepiped, or a cube, each having a height and diameter of several millimeters (mm) or hundreds of millimeters (mm). However, the porous ceramic material is not limited thereto, and various types of porous ceramic materials that can be used by those skilled in the art may be used.

The porous ceramic material may further include an alkali oxide component. Examples of the alkali oxide component include Li₂O, Na₂O, or K₂O. The porous ceramic material further including the alkali oxide component may maintain the shape of the ozone decomposition catalyst structure even at high temperatures without thermal deformation.

The support 1 has a large specific surface area even compared to a support containing organic materials such as polybenzimidazole or polyamide, and may show excellent α-MnO₂ catalytic activity. In addition, the support 1 may retain the shape thereof even by external environments such as strong acids, high temperature, and strong wind.

The amount of the nano manganese oxide 3 may be from 1 part by weight to 100 parts by weight based on 100 parts by weight of the support 1. In an embodiment, the amount of the nano manganese oxide 3 may be, based on 100 parts by weight of the support 1, from 1 part by weight to 80 parts by weight, from 1 part by weight to 60 parts by weight, from 1 part by weight to 40 parts by weight, or from 1 part by weight to 20 parts by weight.

Within the amount ranges of the nano manganese oxide 3, a coating solution containing the nano manganese oxide 3 may be easily applied to the support 1 in a sufficient amount for catalytic activity. In the case where the support 1 is porous, pores or openings may not be blocked.

The ozone decomposition catalyst structure 10 may be a binder-free structure. A sufficient amount of a binder is required to coat (nano) manganese oxide on an organic material support such as a commonly used fiber aggregate, and the catalytic activity of ozone decomposition may be reduced. In addition, since organic material supports such as fiber aggregates have flexible properties, the shapes thereof are changed due to external environments such as strong acids, high temperatures, and strong winds. Accordingly, there is a need to provide a separate design to fix the supports. The ozone decomposition catalyst structure 10 according to an embodiment may be fixed in the pores and on surfaces inside the support without a binder, so that the catalytic activity of ozone decomposition can be further increased.

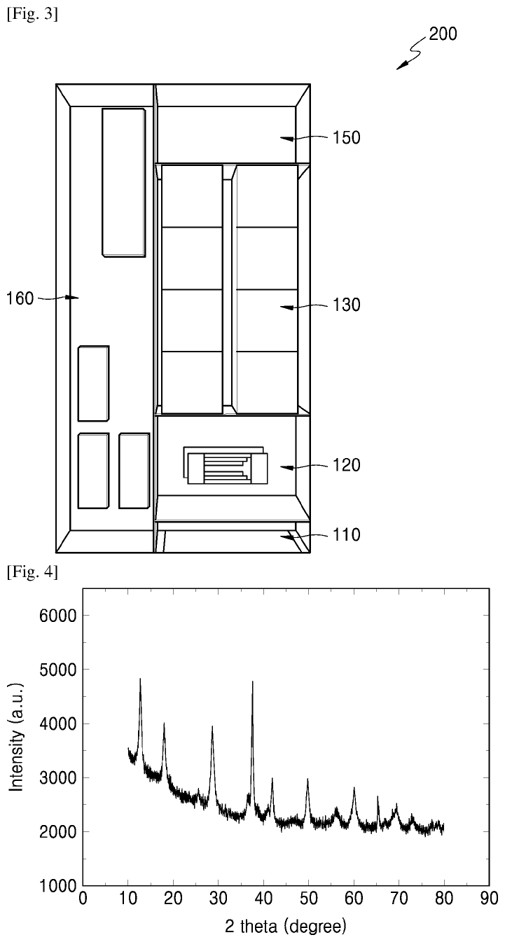

FIG. 2 shows a schematic diagram of an air-cleaning device 20 according to an embodiment. FIG. 3 shows a schematic diagram of the air-cleaning device 200 according to an embodiment.

Referring to FIGS. 2 and 3, the air-cleaning device 20 or 200 according to an embodiment may include an air inlet 14 or 110, the ozone generating unit 11 or 120, an ozone decomposition unit 12 or 130, and an air outlet 15 or 150.

The air inlet 14 or 110 is an area through which air is introduced from the outside.

The ozone generating unit 11 or 120 may include one or more ozone generators of a corona discharge ozone generator or a cold plasma ozone generator. In an embodiment, the ozone generating unit 11 or 120 may be a corona discharge ozone generator. The ozone generating unit 11 or 120 may further improve the performance of reducing harmful gases including ethylene and harmful microorganisms by appropriately controlling voltage, current, or power.

In addition, the ozone generating unit 11 or 120 may further include an electric energy storage unit and a charge controller. The electric energy storage unit is electrically connected to an electric generator and stores the electric energy produced therefrom. The charge controller is configured to be combined with the electric generator and the electric energy storage unit and is configured to control the charging and discharging of electricity in the electric generator and the electric energy storage unit. An example of the electric generator may be a horizontal axis turbine, and an example of the electric energy storage unit may be a battery. The air inlet 14 or 110 and the ozone generating unit 11 or 120 may be physically connected to each other through a pipe (not illustrated) or an air passage that guides the air flow to the ozone generating unit 11 or 120 (not illustrated). Alternatively, the air inlet 14 or 110 may be fixedly arranged on one surface of the ozone generating unit 11 or 120.

The ozone decomposition unit 12 or 130 includes the ozone decomposition catalyst structure 10 located therein.

The air outlet 15 or 150 is an area through which internal air is discharged to the outside.

The air-cleaning device 20 or 200 enables the inflow and outflow of air in one direction.

A fan may be provided in at least one of the air inlet 14 or 110 and the air outlet 15 or 150. In an embodiment, a fan may be fixedly arranged on the air inlet 14 or 110 and the air outlet 15 or 150 or in a separate area.

The air-cleaning device 20 or 200 may additionally include an electric device unit 160, a compressor, or a pump.

The harmful gas may include organic-inorganic harmful gas including ethylene, ammonia, acetaldehyde, or a combination thereof. The harmful microorganisms may include fungi, E. coli, Pseudomonas aeruginosa, Staphylococcus, or a combination of these.

An air-cleaning method for reducing harmful gases including ethylene and harmful microorganisms according to an embodiment includes: a first step of reducing harmful gas including ethylene and harmful microorganisms in air by using at least one ozone generator of a corona discharge ozone generator or a cold plasma ozone generator; and a second step of decomposing ozone generated in the first step, by using an ozone decomposition catalyst structure including a support and a nano manganese oxide located in at least a portion of the inside and surface of the support, wherein the nano manganese oxide may include at least one of α-MnO₂, β-MnO₂, γ-MnO₂, δ-MnO₂, or amorphous MnO₂, and the nano manganese oxide may have a shape selected from a nanorod shape, a nanofiber shape, a nano sea-urchin shape, or a nanoflower shape.

The air-cleaning method may effectively reduce or remove harmful gases, including ethylene, harmful microorganisms, and ozone existing outside or/and inside a device without exchanging filters. Furthermore, the air-cleaning method may maintain the freshness of fruits and vegetables in a closed space such as a reservoir. The ozone generator, the support, the nano manganese oxide, the ozone decomposition catalyst structure, the composition and shape of the nano manganese oxide, harmful gas, or harmful microorganisms are the same as described above, and thus, detailed description thereof will be omitted.

An air-cleaning system according to an embodiment may include an air-cleaning device including the ozone decomposition catalyst structure described above. The air-cleaning system may further include a sensor, or a temperature controller, when needed.

Hereinafter, Examples and Comparative Examples of the present disclosure will be described. However, the following example is only an example of present disclosure, and the present disclosure is not limited to the following examples.

### EXAMPLES

### Preparation Example 1

### Preparation of Ozone Decomposition Catalyst Structure

13.1 g of MnCl₂·4H₂O and 21.7 g of KMnO₄ were added to 250 ml of water and stirred together to obtain a mixed solution. 250 mL of the mixed solution was heated to the temperature of 220° C. for 2 hours in a hydrothermal reactor, caused to react at a temperature of 220° C. for 12 hours, and then filtered. Then, the obtained precipitate was dried at a temperature of 100° C. for 2 hours to obtain a bulky α-MnO₂. A α-MnO₂-containing solution (a solid content of about 10%) in which the bulky α-MnO₂ was dispersed in water, was milled. As a result, an α-MnO₂ dispersion containing α-MnO₂ nanorods having an aspect ratio (diameter: length) of about 1:40 was obtained.

A porous cordierite monolith (50×50 mm/200 cpsi, manufactured by Ceracomb Co., Ltd.) having the shape of cylinder, containing 50% or more of MgO, SiO₂, and Al₂O₃ components, and having the diameter of 50 mm×the height of 50 mm was prepared. The porous cordierite monolith was dipped in the α-MnO₂ dispersion and then dried to prepare an ozone decomposition catalyst structure in which the α-MnO₂ nanorod is coated the inside and surface of the porous cordierite monolith, thereby completing the manufacture of the ozone decomposition catalyst structure as illustrated in FIG. 1.

At this time, the amount of the α-MnO₂ catalyst was 10 parts by weight based on 100 parts by weight of the porous cordierite monolith.

### Preparation Example 2

### Preparation of Ozone Decomposition Catalyst Structure

13.1 g of MnCl₂·4H₂O, 21.7 g of KMnO₄, and 12.2 g of CuCl₂ were added to 250 ml of water and stirred together to obtain a mixed solution. 250 mL of the mixed solution was heated to the temperature of 220° C. for 2 hours in a hydrothermal reactor, caused to react at a temperature of 220° C. for 12 hours, and then filtered. Then, the obtained precipitate was dried at a temperature of 100° C. for 2 hours to obtain a bulky α-MnO₂ doped with 5 wt % of copper. A α-MnO₂-containing solution (a solid content of about 10%) in which the bulky α-MnO₂ doped with 5 wt % of copper was dispersed in water, was milled. As a result, an α-MnO₂ dispersion containing α-MnO₂ particles doped with 5 wt % of spherical copper having an average diameter of about 30 nm (D50), was obtained.

The porous cordierite monolith (50×50 mm/200 cpsi, manufactured by Ceracomb Co., Ltd.) having the shape of cylinder, containing 50% or more of MgO, SiO₂, and Al₂O₃ components, and having the diameter of 50 mm×the height of 50 mm was dipped in the α-MnO₂ dispersion and dried, thereby obtaining the ozone decomposition catalyst structure illustrated in FIG. 1 in which α-MnO₂ particles doped with 5 wt % of copper were coated inside of the porous cordierite monolith and on the surface thereof.

At this time, the amount of the α-MnO₂ catalyst which was doped with 5 wt % of copper, was 15 parts by weight based on 100 parts by weight of the porous cordierite monolith.

### Preparation Example 3

### Preparation of Ozone Decomposition Catalyst Structure

The bulky α-MnO₂ prepared according to Preparation Example 1 and multi-walled carbon nanotubes (MWCNT, manufactured by Kumho Petrochemical Co., Ltd.) were mechanically mixed at the weight ratio of 3:1 to obtain a mixture. The bulky α-MnO and MWCNT-containing solution (solid content of about 10%) in which the mixture was dispersed in a mixed solvent of water and ethanol (the volumetric ratio of 3:7), was milled. As a result, a mixed dispersion containing α-MnO₂ nanorods and MWCNT having an aspect ratio (diameter: length) of about 1:40, was obtained.

A cylindrical porous aluminum monolith (manufactured by Foshan Jinbaishi TEch. (China)) was prepared. The cylindrical porous aluminum monolith was dipped in the α-MnO₂ nanorod and MWCNT-containing mixed dispersion, and then, dried, thereby obtaining the ozone decomposition catalyst structure, illustrated in FIG. 1, in which α-MnO₂ nanorod and MWCNT were coated inside of the porous aluminum monolith and on the surface thereof.

At this time, the amount of the α-MnO₂ and MWCNT-containing catalyst was 10 parts by weight based on 100 parts by weight of the porous aluminum monolith.

### Preparation Example 4

### Preparation of Ozone Decomposition Catalyst Structure

16.0 g of MnCl₂·4H₂O and 12.8 g of K₂CrO₇ were added to 250 ml of water and stirred together to obtain a mixed solution. A porous cordierite monolith (50×50 mm/200 cpsi, manufactured by Ceracomb Co., Ltd.) having the shape of cylinder, containing 50% or more of MgO, SiO₂, and Al₂O₃ components, and having the diameter of 50 mm×the height of 50 mm was dipped in the mixed solution. Thereafter, the porous cordierite monolith was heated to 60° C. for 30 minutes in a hydrothermal reactor and maintained for 24 hours. The porous cordierite monolith was washed three times with distilled water and dried, thereby obtaining an ozone decomposition catalyst structure coated with α-MnO₂ nano sea-urchin with an aspect ratio (diameter: length) of about 1:5 and a nanosheet having a thickness of about 40 nm which were present inside of the porous cordierite monolith and on the surface thereof.

At this time, the amount of the α-MnO₂ catalyst was 5 parts by weight based on 100 parts by weight of the porous cordierite monolith.

### Preparation Example 5

### Preparation of Ozone Decomposition Catalyst Structure

6.0 g of Mn(COOH)₂·4H₂O and 11.0 g of K₂S₂O₈ were added to 250 ml of water and stirred together to obtain a mixed solution. The mixed solution was heated to 55° C. for 30 minutes in a water bath and maintained for 12 hours to cause a reaction. Then, the obtained precipitate was filtered and dried at 100° C. for 2 hours to obtain a bulky catalyst in which α-MnO₂ nanofibers having an aspect ratio (diameter: length) of about 1:1000 was mixed with α-MnO₂ nanoflowers having a diameter of about 400 nm.

The bulky catalyst was put in water and dispersed therein (a solid content of about 10%), and the resultant solution was milled. As a result, a catalyst powder dispersion was obtained in which α-MnO₂ nanofibers having an aspect ratio (diameter: length) of about 1:1000 was mixed with α-MnO₂ nanoflowers having a diameter of about 400 nm.

A porous cordierite monolith (50×50 mm/200 cpsi, manufactured by Ceracomb Co., Ltd.) having the shape of cylinder, containing 50% or more of MgO, SiO₂, and Al₂O₃ components, and having the diameter of 50 mm×the height of 50 mm was prepared. The porous cordierite monolith was dipped in the dispersion and then dried to prepare an ozone decomposition catalyst structure, illustrated in FIG. 1, in which α-MnO₂ was coated the inside of the porous cordierite monolith and on the surface thereof.

At this time, the amount of the α-MnO₂ catalyst was 10 parts by weight based on 100 parts by weight of the porous cordierite monolith.

### Comparative Preparation Example 1

### Ozone Decomposition Catalyst

Manganese granules having an average particle diameter (D50) of about 3 mm were prepared using an ozone decomposition catalyst.

### Example 1

### Manufacture of Air-Cleaning Device

The first reaction chamber 120, which is an ozone generating unit in which an air inlet (including a fan, 110) was provided on one side thereof, had a corona discharge plate (power: 60 W, manufactured by Ozonetech Co., Ltd.) placed in the center thereof. In the second reaction chamber 130, which is an ozone decomposition unit, eight ozone decomposition catalyst structures manufactured according to Preparation Example 1 were placed therein. The first reaction chamber 120 and the second reaction chamber 130 were connected via a connecting pipe (not illustrated), which was used as a passage for introducing the ozone-containing air generated from the first reaction chamber 120 into the second reaction chamber 130. An air outlet 150 was provided on one side of the second reaction chamber 130, and through the air outlet 150, cleaned air was discharged. A fan (3214JH, manufactured by ebm-papst Inc.) was provided on the air outlet 150, thereby completing manufacture of an air-cleaning device. The corona discharge plate inside the first reaction chamber 120 and the fan provided on the air outlet 150 were each connected to a power source. Air was allowed to flow in one direction from the air inlet 110 to the first reaction chamber 120, the second reaction chamber 130, and the air outlet 150.

### Examples 2 to 4

### Manufacture of Air-Cleaning Device

An air-cleaning device was manufactured in the same manner as in Example 1, except that 8 ozone decomposition catalyst structures manufactured according to Preparation Examples 2 to 4 were arranged inside the second reaction chamber 130, which is an ozone decomposition unit.

### Comparative Example 1

### Manufacture of Air-Cleaning Device

An air-cleaning device was manufactured in the same manner as in Example 1, except that the ozone decomposition catalyst structure manufactured according to Comparative Preparation Example 1 was arranged inside the second reaction chamber 130, which is an ozone decomposition unit.

### Comparative Example 2

### Manufacture of Air-Cleaning Device

An air-cleaning device was manufactured in the same manner as in Example 1, except that the total of six UV-C lamps (wavelength of 254 nm: wavelength of 185 nm =9:1, power: 11W, manufactured by light sources Inc.) were provided at the center region of the first reaction chamber 120, which is an ozone generating unit.

### Analysis Example 1

### X-Ray Diffraction (XRD) Analysis

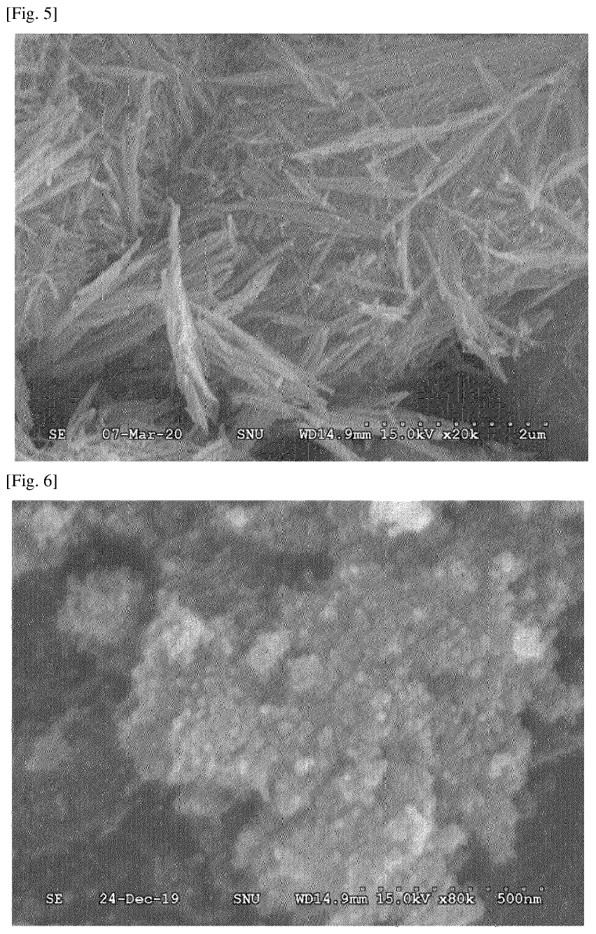

XRD experiment was performed on the α-MnO₂ nanorod catalyst of the ozone de-composition catalyst structure prepared according to Preparation Example 1. In the XRD experiment, the powder obtained by filtering and drying the dispersion containing α-MnO₂ nanorods, was measured for XRD. The results are shown in FIG. 4. As an XRD analyzer, a Rigaku RINT2200HF+ diffractometer using CuKαradiation (1.540598Å) was used.

Referring to FIG. 4, distinct peaks appeared when the diffraction angle 2θ of the α-MnO₂ nanorod catalyst of the ozone decomposition catalyst structure prepared according to Preparation Example 1 was about 13°, about

18°, about 29°, about 37°, and about 60° As a result, it can be seen that the α-MnO₂ nanorod catalyst of the ozone decomposition catalyst structure was pure α-MnO₂.

### Analysis Example 2

### SEM Image of Ozone Decomposition Catalyst

Scanning electron microscope (SEM) photographs were taken of the ozone decomposition catalyst of the ozone decomposition catalyst structure prepared according to Preparation Examples 1 to 5. The results are shown in FIGS. 5 to 9.

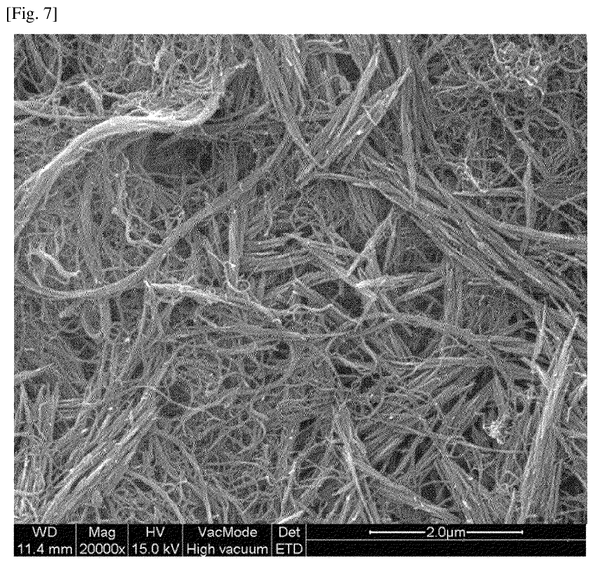

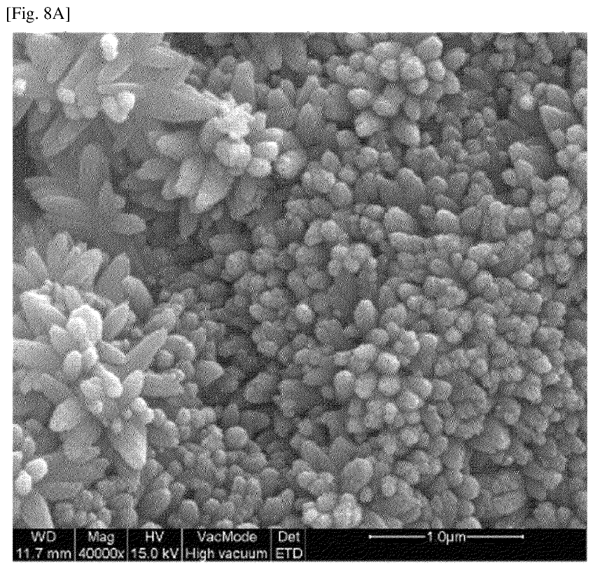

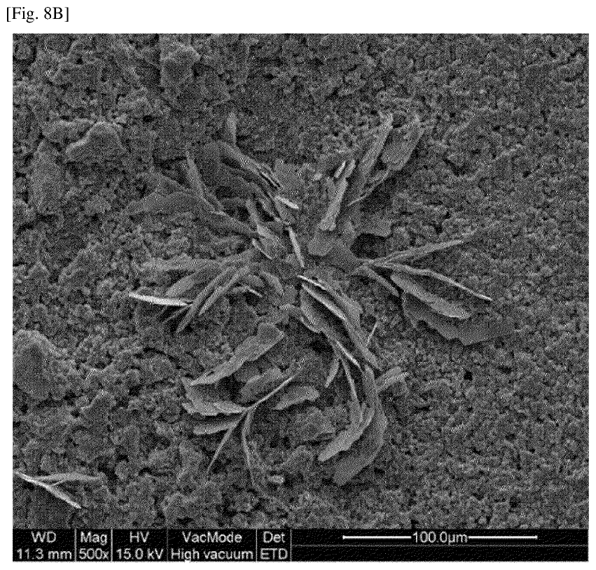

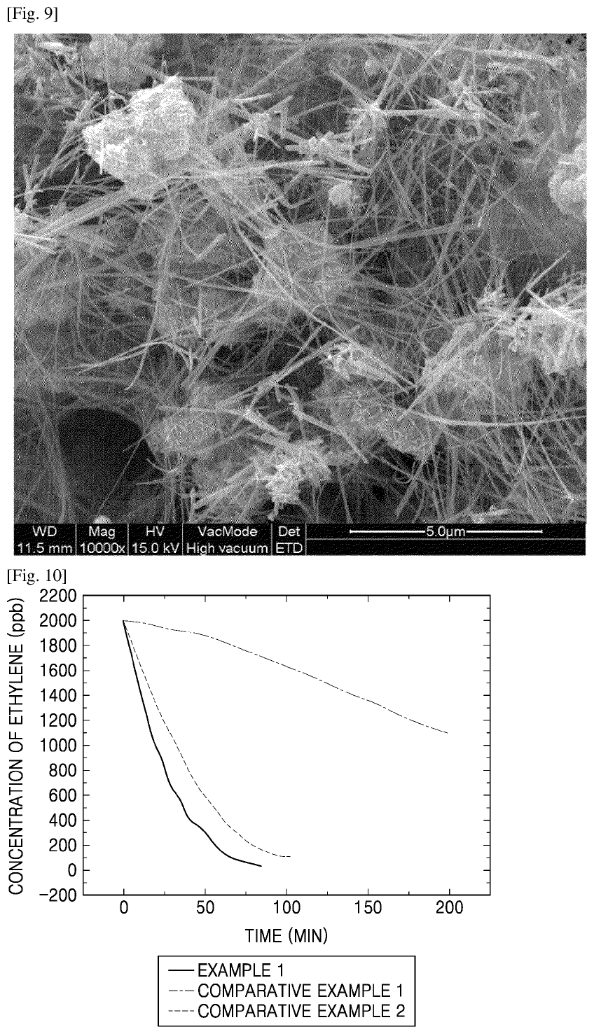

As shown in FIG. 5, the ozone decomposition catalyst of Preparation Example 1 had a nanorod shape having an aspect ratio (diameter: length) of about 1:40. As shown in FIG. 6, the ozone decomposition catalyst of Preparation Example 2 was spherical particles having an average diameter (D50) of about 30 nm. As shown in FIG. 7, in the ozone decomposition catalyst of Preparation Example 1, α-MnO₂ having a nanorod shape having an aspect ratio (diameter: length) of about 1:40 and MWCNT co-existed. As shown in FIGS. 8A and 8B, in the ozone decomposition catalyst of Preparation Example, a nano-sea urchin shape having an aspect ratio (diameter: length) of about 1:5 and a nanosheet shape having a thickness of about 40 nm co-existed. As shown in FIG. 9, in the ozone decomposition catalyst of Preparation Example 5, a nano-sea urchin shape having an aspect ratio (diameter: length) of about 1:5 and a nanosheet shape having a thickness of about 40 nm co-existed.

### Evaluation Example 1

### Ethylene Gas Reduction Performance Evaluation

A chamber having the size of 2 m×1 m×1 m with temperature control was prepared. The temperature inside the chamber was set to 15° C. and the relative humidity thereof was maintained at 50% to 60%. A certain concentration of ethylene was injected into the chamber.

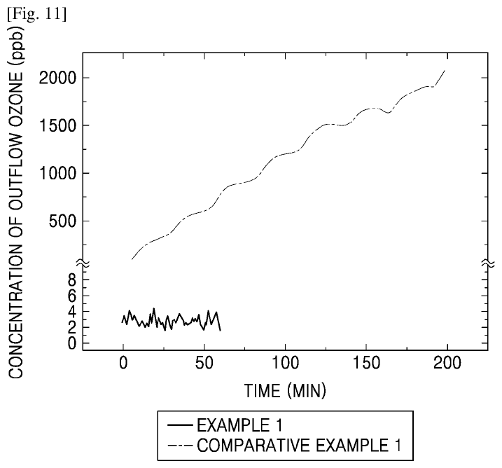

An evaluation result of ethylene gas reduction performance of air-cleaning devices manufactured according to Example 1, Comparative Example 1, and Comparative Example 2 was performed. In order to evaluate the ethylene gas reduction performance, the concentration of ethylene remaining in the chamber space according to the operating time of the air-cleaning device was measured. The concentration of ethylene was measured at intervals of 6 minutes through gas chromatography (manufactured by Umwelttechnik MCZ GmbH) capable of auto-sampling. Some of the results are shown in Table 1 and FIG. 10.

TABLE 1

| | Concentration of ethylene (ppb) | | |
|---|---|---|---|
| Elapsed time | | Comparative | Comparative |
| (min) | Example 1 | Example 1 | Example 2 |
| 0 | 2000 | 2000 | 2000 |
| 30 | 866 | 1949 | 1209 |
| 60 | 245 | 1848 | 629 |
| 90 | 35 | 1704 | 170 |

Referring to Table 1 and FIG. 10, it was confirmed that, in the air-cleaning device manufactured according to Example 1, when the initial concentration of ethylene gas was 2000 ppb, the ethylene gas was reduced to 50 ppb or less after about 1.5 hours. In comparison, in the air-cleaning device manufactured according to Comparative Example 1, when the initial concentration of ethylene gas was 2000 ppb, the ethylene gas was 1704 ppb after about 1.5 hours, and the ethylene gas was 1200 ppb or more after about 3 hours. In the air-cleaning device manufactured according to Comparative Example 2, when the initial concentration of ethylene gas was 2000 ppb, the ethylene gas was 170 ppb after about 1.5 hours, and the ethylene gas was 50 ppb or more after about 2 hours. From these results, it was confirmed that, compared with the air-cleaning devices manufactured according to Comparative Examples 1 and 2, the concentration of ethylene gas of the air-cleaning device manufactured according to Example 1 was reduced faster. This shows that the ethylene gas reduction performance has been improved.

### Evaluation Example 2

### Evaluation of Ozone Decomposition Performance

The ozone decomposition performance evaluation of the air-cleaning devices manufactured by Example 1 and Comparative Example 1 was performed in the same environment as the chamber described in Evaluation Example 1. To evaluate ozone decomposition performance, the concentration of outflow ozone over the operating time of the air-cleaning device was measured. The concentration of outflow ozone was measured using an ultraviolet absorption method (Model 202, manufactured by 2B technology Inc.). The results are shown in FIG. 11.

Referring to FIG. 11, the air-cleaning device manufactured according to Example 1 maintained the concentration of outflow ozone at about 3 ppb even after about 80 minutes elapsed. In comparison, in the air-cleaning device manufactured according to Comparative Example 2, the concentration of outflow ozone continuously increased to 2000 ppb until about 200 minutes elapsed. From these results, it was confirmed that, compared with the air-cleaning devices manufactured according to Comparative Example 1, the air-cleaning device manufactured according to Example 1 maintained the lower concentration of outflow ozone for a long time. This shows that the ozone gas decomposition performance has been improved.

In the above, embodiments have been described in detail with reference to the accompanying drawings, but the present disclosure is not limited to the related examples. To those with ordinary knowledge in the technical field to which the present disclosure belongs, it is clear that their thoughts affect various changes or modifications within the scope of the technical concept described in the claims. Even these changes and modifications are understood to belong to the technical range of the present disclosure.

1. An air-cleaning device for reducing harmful gases including ethylene and harmful microorganisms, the air-cleaning device comprising:
an air inlet for intaking air from the outside;
an ozone generating unit in which at least one ozone generator of a corona discharge ozone generator or a cold plasma ozone generator is located;
an ozone decomposition unit for removing ozone generated in the ozone generating unit, in which a support and an ozone decomposition catalyst structure including a nano manganese oxide are located; and
an air outlet for outflowing the internal air to the outside, wherein
the nano manganese oxide is located on at least a portion of the inside and the surface of the support,
the nano manganese oxide includes at least one of α-MnO₂, β-MnO₂, γ-MnO₂, δ-MnO₂, or amorphous MnO₂, and
the nano manganese oxide has a shape selected from a nanorod shape, a nanofiber shape, a nano sea-urchin shape, a nanoflower shape, or a nanosheet shape.

2. The air-cleaning device of claim 1, wherein
the nano manganese oxide comprises at least one of α-MnO₂ or β-MnO₂, and
α-MnO₂ or β-MnO₂ has a nanorod shape, a nanofiber shape, or a nano sea-urchin shape, and an aspect ratio of 1:5 to 1:1000.

3. The air-cleaning device of claim 1, wherein
the nano manganese oxide is β-MnO₂, and
the δ-MnO₂ has a nanoflower shape or a nanosheet shape, and a thickness thereof is from 5 nm to 400 nm.

4. The air-cleaning device of claim 1, wherein
the nano manganese oxide is a crystalline MnO₂ nanoparticle or an amorphous MnO₂ nanoparticle, and
the nanoparticle has a diameter of 1 nm to 500 nm.

5. (canceled)

6. The air-cleaning device of claim 1, wherein the nano manganese oxide further comprises at least one selected from metal oxide, silicon oxide, carbon nanotubes, activated carbon, graphene, or graphene oxide.

7. The air-cleaning device of claim 1, wherein the support is a ceramic material, a metal material, or a combination of these, in the form of a monolith or a foam.

8. The air-cleaning device of claim 1, wherein the ozone decomposition catalyst structure is a binder-free structure.

9. (canceled)

10. (canceled)

11. The air-cleaning device of claim 1, wherein the harmful gas comprises an organic-inorganic harmful gas including ethylene, ammonia, acetaldehyde, or a combination of these.

12. The air-cleaning device of claim 1, wherein the harmful microorganisms comprises fungi, E. coli, Pseudomonas aeruginosa, Staphylococcus, or a combination of these.

13. An air-cleaning method for reducing harmful gases including ethylene and harmful microorganisms, the air-cleaning method comprising:
a first step of reducing harmful gas including ethylene and harmful microorganisms in air by using at least one ozone generator of a corona discharge ozone generator or a cold plasma ozone generator; and
a second step of decomposing ozone generated in the first step, by using an ozone decomposition catalyst structure including a support and a nano manganese oxide located in at least a portion of the inside and surface of the support, wherein
the nano manganese oxide includes at least one of α-MnO₂, β-MnO₂, γ-MnO₂, δ-MnO₂, or amorphous MnO₂, and
the nano manganese oxide has a shape selected from a nanorod shape, a nanofiber shape, a nano sea-urchin shape, a nanoflower shape, or a nanosheet shape, a nanosphere shape.

14. The air-cleaning method of claim 13, wherein
the manganese oxide comprises at least one of α-MnO₂ or β-MnO₂, and
α-MnO₂ or β-MnO₂ has a nanorod shape, a nanofiber shape, or a nano sea-urchin shape, and an aspect ratio of 1:5 to 1:1000.

15. The air-cleaning method of claim 13, wherein
the nano manganese oxide is β-MnO₂, and
the δ-MnO₂ has a nanoflower shape or a nanosheet shape, and a thickness thereof is from 5 nm to 400 nm.

16. The air-cleaning method of claim 13, wherein
the nano manganese oxide is a crystalline MnO₂ nanoparticle or an amorphous MnO₂ nanoparticle, and
the diameter of the nanoparticles is from 1 nm to 500 nm.

17. (canceled)

18. The air-cleaning method of claim 13, wherein the nano manganese oxide further comprises at least one selected from metal oxide, silicon oxide, carbon nanotubes, activated carbon, graphene, or graphene oxide.

19. The air-cleaning method of claim 13, wherein the support includes a ceramic material, a metal material, or a combination of these, in the form of a monolith or foam.

20. The air-cleaning method of claim 13, wherein the ozone decomposition catalyst structure is a binder-free.

21. The air-cleaning method of claim 13, wherein the harmful gas comprises an organic-inorganic harmful gas including ethylene, ammonia, acetaldehyde, or a combination of these.

22. The air-cleaning method of claim 13, wherein the harmful microorganisms comprise fungi, E. coli, Pseudomonas aeruginosa, Staphylococcus, or a combination of these.

23. An air-cleaning system including the air-cleaning device of claim 1.
